(19) United States
(12) Patent Application Publication
(10) Pub. No.: US 2025/0340819 A1
(43) Pub. Date: Nov. 6, 2025

(54) DEVICES FOR GENERATING CAVITATIONS IN TISSUE

(71) Applicant: The Charles Stark Draper Laboratory, Inc., Cambridge, MA (US)

(72) Inventors: Tyler Gerhardson, Cambridge, MA (US); Vishal Tandon, Cambridge, MA (US); Ernest Kim, Cambridge, MA (US); Jennifer Walker, Cambridge, MA (US)

(21) Appl. No.: 19/196,257

(22) Filed: May 1, 2025

Related U.S. Application Data
(60) Provisional application No. 63/642,246, filed on May 3, 2024.

(51) Int. Cl. C12M 1/42; C12M 1/32; C12M 3/00

(52) U.S. Cl. CPC C12M 35/02; C12M 21/08; C12M 23/12

(57) ABSTRACT

A system to simulate injury in tissue culture may include a plate configured to contain tissue culture and defining a plurality of wells, a transducer configured to couple with the plate to create a coupling path between the plate and the tissue culture within the plate to generate cavitations within the tissue culture, a medium coupling the transducer to the plate, and an electronic driver configured to emit a waveform to drive the transducer and allow assessment of the cavitations.

### CROSS-REFERENCED TO RELATED APPLICATION

This application claims the benefit of U.S. provisional application Ser. No. 63/642,246 filed May 3, 2024, the disclosure of which is hereby incorporated in its entirety by reference herein.

### TECHNICAL FIELD

Aspects of the disclosure generally relate to devices for generating cavitations in tissue.

### BACKGROUND

Blast wave exposure from explosions can cause severe injuries to various human tissues, especially the brain. A major hypothesis is that these injuries stem from cavitation-tiny vapor bubbles that form and violently collapse due to pressure changes in body fluids like cerebrospinal fluid (CSF) when a blast wave hits. This process can damage delicate tissues such as nerves. Studying the effects of explosions may be difficult.

### SUMMARY

A system to simulate injury in tissue culture may include a plate configured to contain tissue culture and defining a plurality of wells, a transducer configured to couple with the plate to create a coupling path between the plate and the tissue culture within the plate to generate cavitations within the tissue culture, a medium coupling the transducer to the plate, and an electronic driver configured to emit a waveform to drive the transducer and allow assessment of the cavitations.

A system to simulate injury in tissue culture may include a plate configured to contain tissue culture and defining a plurality of wells, a transducer arranged with the plate creating a coupling path between the plate and the tissue culture within the plate; a medium coupling the transducer to the plate; and a driving system configured to emit a waveform to drive the transducer and allow assessment of injury to the tissue culture.

### BRIEF DESCRIPTION OF THE DRAWINGS

FIG. 1 illustrates an example microphysiological system (MPS).

FIG. 2 illustrates a side view of the stage assembly of FIG. 1.

FIG. 3 illustrates an example plate 120 of the system 100.

FIG. 4 illustrates a simplified chart of the reflection within the hole.

FIG. 5 illustrates a side view of the transducer and reflection fiducial.

FIG. 6 illustrates another example plate of FIG. 1 and a cross-sectional view of an example well of the plate relative to the transducer.

FIG. 7 illustrates a cross-sectional view of the well of FIG. 6 without the transducer and shows the geometric relationship created by the transducer.

FIG. 8 illustrates an example chart for the simulated focal pressure showing distance (mm) vs. pressure (Mpa).

FIG. 9 illustrates a cross-sectional view of the coupling and targeting arrangement of FIG. 6.

FIG. 10 illustrates a top view of the arrangement of FIG. 9 including the MPS.

FIG. 11 illustrates a cross-sectional view an example well of the plate relative to the transducer similar to FIG. 6 and includes the plate reflection and cavitation emission.

FIG. 12 illustrates an example voltage production of the transducer over time.

FIG. 13 illustrates an example voltage and power over time of the set up of FIG. 12.

FIG. 14 illustrates a cross-section view of a transducer under excitation, having cavitation.

FIG. 15 illustrates a view of the transducer being aligned and integrated with the in-vitro platform to generate cavitations within a single well of the platform.

FIG. 16 illustrates a view of cavitation generated hole.

FIG. 17 illustrates an example voltage over time of an injury confirmation in situ.

FIG. 18 illustrates a top view of an example well.

FIG. 19 illustrates a top view of a sample.

FIG. 20 illustrates a side view of the sample of FIG. 19.

FIG. 21A illustrates a model with 120 cavitation events.

FIG. 21B illustrates a model with 1,200 cavitation events.

FIG. 21C illustrates a model with 12,000 cavitation events.

FIG. 22 illustrates a series of focal zone images where the cavitation emissions received by the transducer indicative of the absence or presence of injury.

FIG. 23 illustrates a series of signal received by the transducer indicative of the absence or presence of injury.

FIG. 24 illustrates an example configuration to deliver pulses of cavitation uniformly across area of tissue using continuous raster.

FIG. 25 illustrates an example configuration to delivery pulses of cavitation uniformly across area of tissue using discrete points on a grid as opposed to continuous raster scanning.

FIG. 26 illustrates a cross-sectional view of an example transducer.

FIG. 27 illustrates a cross-sectional view of another example transducer.

FIG. 28 illustrates an example chart for a LDH assay blast injury showing absorbance over time.

FIG. 29 illustrates an example chart for Glutamate showing absorbance over time.

FIG. 30 illustrates an example chart for GFAP showing concentration over time.

FIG. 31 illustrates an example chart for NSE showing concentration over time.

FIG. 32 illustrates an example chart for S100B showing concentration over time.

FIG. 33 illustrates an example chart for UCHL showing concentration over time.

FIG. 34 illustrates an example chart for NF-H showing concentration over time.

FIG. 35 illustrates example images of neuron structures at various pulse repetition frequencies and doses over time.

FIG. 36 illustrates an example chart for the total ROI analyzed for neuron networks in scaffolds exposed to blast treatments over frequency.

FIG. 37 illustrates an example chart for the area covered by neuron networks over pulse repetition frequency.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

The impingement of a blast wave on the body from nearby explosions could cause severe injury to living bodies, including injury to many tissue types including brain, gastrointestinal and pulmonary tissue. For example, 50-60% of all traumatic brain injury (TBI) cases are associated with explosions and many cases are attributed to the impingement of a blast wave on the body. A leading hypothesis for blast wave associated injury is that cavitation produced by the interaction of the blast wave with the body is the primary cause of these injuries. For example, the interaction of a blast wave with the skull can cause the skull to flex, leading to high and low pressures zones in the adjacent cerebral spinal fluid (CSF). The low-pressure zones produce cavitation within the CSF and surrounding brain tissue which strains delicate nervous tissue and causes injury.

However, studying the effects of blast waves on different tissue types is challenging. This is in part due to the lack of appropriate biological and blast wave models. Traditional cell culture is too simplified (single cell types, lack of important microenvironmental cues) while the complexity of animal models makes it challenging to recreate the anatomical dependent mechanisms involved and may not accurately reflect human physiology. To this end, complex in vitro models such as microphysiological systems (MPS) paired with an accurate blast model is attractive to understanding the impacts of blast waves at the cellular level to elucidate new relevant targets for medical counter measures (MCMs).

Disclosed herein is a system that incorporates focused ultrasound (FUS) transducers with a high-throughput MPS platform. The FUS transducers are designed to focus mechanical energy within the tissue layers of the device. When the rarefactional phase of the pressure pulse exceeds ˜−18 to −26 MPa, a cavitation event occurs at the focus with high spatial (<1 mm) and temporal precision (˜3 μs). By this, the system directly generates cavitation within the target tissue to simulate the key mechanism of blast wave induced injury. This circumvents the anatomical considerations associated with modeling blast waves (i.e., skull deformation) and makes the MPS platform highly relevant to studying the effects of blast waves on tissues.

The system includes a (1) the transducer design including a multi-axis positioner, (2) the placement of the transducer relative to the plate, (3) the medium coupling to transducer to the plate, (4) the electronic pulse used to drive the transducer and (5) the design of the plate. The system allows for cavitation measurements without visual confirmation by using the voltage signals. Specific wells, each containing tissue within the plate are targeted to generate localized cavitation injuries. The system also provides for a control mechanism for sending and receiving signals, including controlling the electronic pulses to drive the transducer.

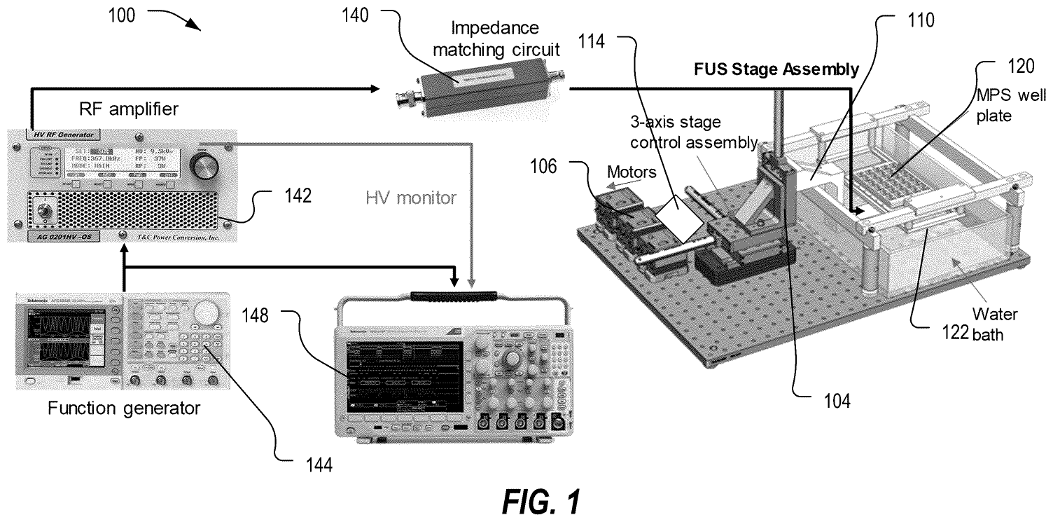

FIG. 1 illustrates a perspective view of an example microphysiological system (MPS) 100 (referred to herein as MPS 100 and system 100). An MPS 100 may be a multi-cellular in vitro system for modeling tissues or organs of an animal or human by mimicking the physiological aspects of the tissues or organ. The MPS 100 may include a FUS stage assembly 102.

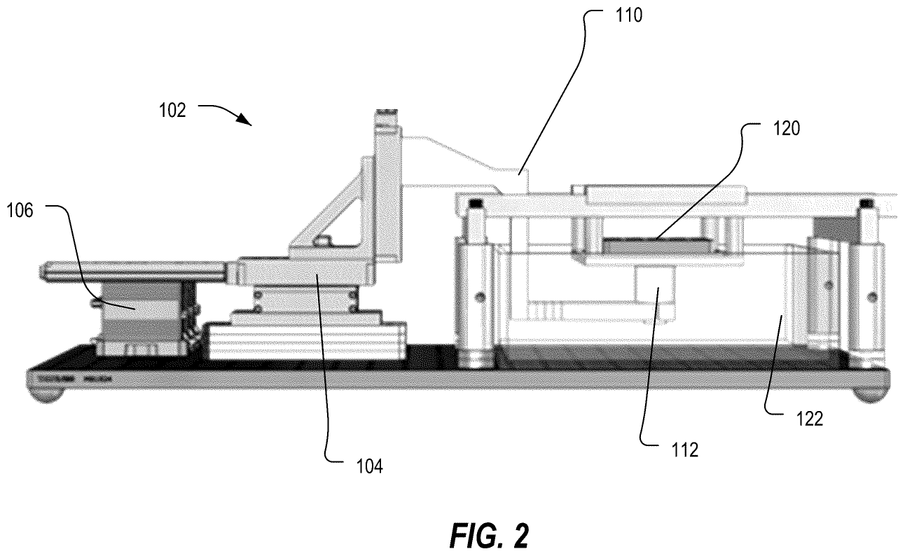

FIG. 2 illustrates a side view of the stage assembly 102 of FIG. 1. Referring to FIGS. 1 and 2, the stage assembly 102 may include a multi-axis, or three-axis control assembly 104 driven by a plurality of motors 106. A transducer arm 110 may be coupled to and extending from the control assembly 104 and hold a transducer 112 (not visible in FIG. 1) on the distal end thereof. The stage assembly 102 may include a plate 120 arranged within a reservoir 122. The transducer arm 110 may extend into the reservoir 122 and under the plate 120. The reservoir 122 may hold a coupling medium such as a fluid, solid or gel. In one example, the medium may be water.

The transducer 112 may extend into the medium. The transducer 112 may be a focused ultrasound (FUS) transducer such as a piezoelectric transducer. The transducer 112 is designed to emit sound waves into the medium. The waves may cause rapid pressure changes, leading to the formation and collapse of vapor bubbles. This is known as cavitation. This may simulate the explosive blasts.

The system 100 may include a controller 114 configured to provide instructions for the processes and method described herein. The controller 114 may be provided with a memory and a central processing unit (CPU). The memory may be used for storing the control software that may be executed by the CPU in completing the processes. The memory may also be used to store information, such as a database or table, and to store data received from the one or more components of the system that may be communicably coupled with the controller 114.

The controller 114 may be operably coupled with one or more components of the system 100 and may include transmitting instructions to the control assembly 104. The controller 114 may be part of the stage assembly 102, but may also be included in the other components of the system 100. Further, more than one controller may be configured to perform part or all of the functions of the system, including monitoring the cavitation in the tissue models, instructing movement of the plate or transducer arm, generating waveforms at the transducer, receiving voltage signals at the transducer, etc.

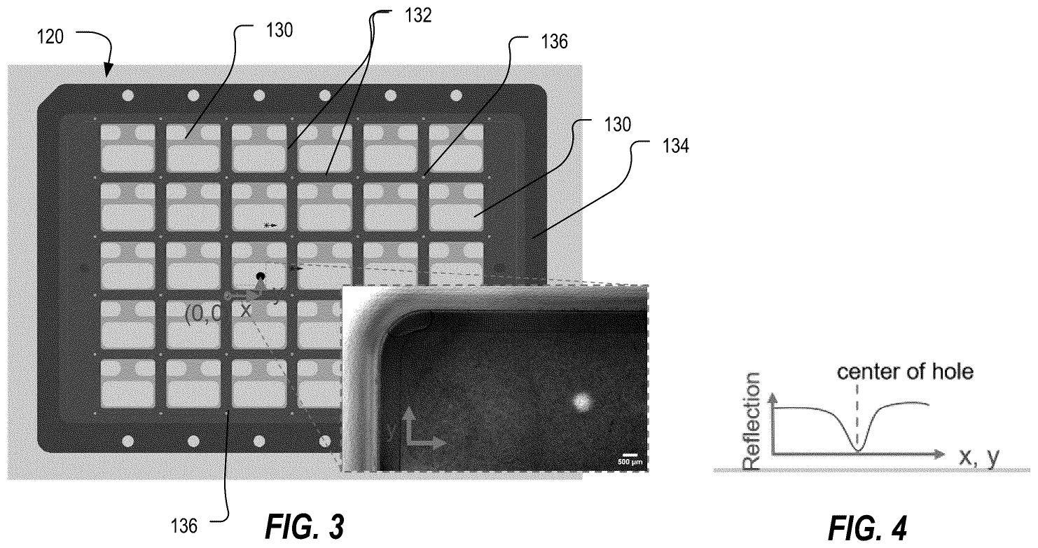

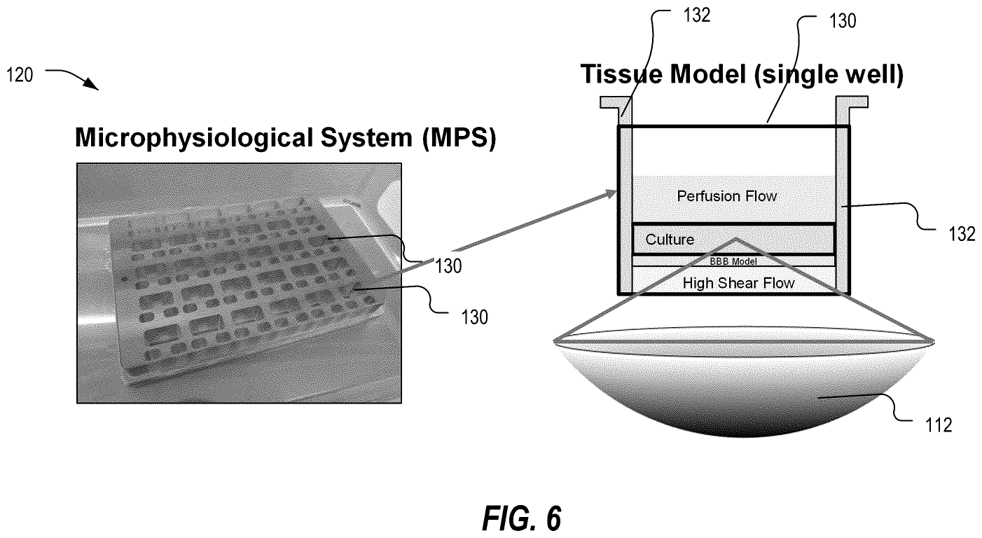

FIG. 3 illustrates an example plate 120 of the system 100. The plate 120, as explained, may be arranged in the reservoir 122. The plate 120 may define a plurality of wells 130. Another example plate 120 can be seen in FIG. 6. The wells 130 are configured to house tissue. The wells 130 may be arranged in rows and columns defined by supports 132 therebetween. The wells 130 may form generally square or rectangular recesses, but in other examples may have circular, oval, or even irregular well shapes. At least one hole 136 may be defined at the corner or apex of the horizontal and vertical supports 132.

A panel 134 may be arrange under the plate 120, or integral with the plate 120. The panel 134 may be formed of metal, and specifically, in one example, may be a highly reflective material such as stainless steel. The holes 136 may be detectable by the transducer 112 via the reflection from the panel 134. The holes 136 may be used to determine the location or precise position of the wells 130 and allow cavitation to be applied to precise positions within the wells. In the example of FIG. 3, the exploded view illustrates an example bright spot. This bright spot may be a lesion generated within a red blood cell phantom. The location of the lesion may be prescribed based on localizing the transducer to the hole 136. The measured location of the lesion corresponds to the target location.

FIG. 4 illustrates a simplified chart of the reflection within the hole 136. In the hole 136, the center of the hole 136 is identified based on the reflection, thus allowing the system to recognize the holes 136 and thus the location of the wells 130.

Referring back to FIG. 3, the panel 134 may also define a group of openings aligned with each of the wells. Each group of openings may form a larger opening arranged adjacent two smaller openings. The panel 134 may be configured to allow for positioning information for each well based on the reflection from the panel 134.

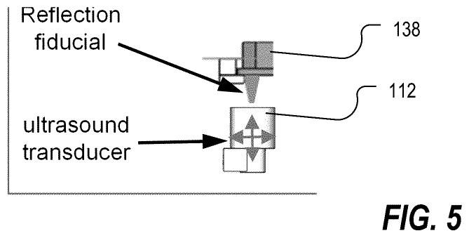

FIG. 5 illustrates a side view of the transducer 112 and reflection fiducial 138. The fiducial 138 is a marker or reference point designed to reflect ultrasound so that the light may be tracked by a sensor. This registration method includes translating the transducer 112 around the fiducial 138 in the x, y, and z axis to maximize reflection signals. While holes 134 may be used to localize a feature and thus focus the transducer relative to the plate and wells, the transducer may also be focused using the reflective feature described here. In this example, the reflective feature may spatially modulate the reflective ultrasound signal to localize the feature.

Referring back to FIGS. 1 and 2, the transducer 112 may be selectively arranged below a selected well 130. The transducer arm 110 may be moved below the selected well 130 via the multi-axis stage assembly 104. As explained, the wells 130 may be identified via reflections from the panel 134 or from the holes 136. The control assembly 104 may navigate the transducer arm 110 based on the identified position of the selected well. In another example, as discussed more in FIG. 10, positioning rails and frames may be used to move the plate 120 over the transducer 112 to align the selected well above the transducer 112. The rails may have features such as magnets or notches that move the well precisely from one well to the next. Further, while the stage assembly 104 is described as being a multi-axis assembly, other possible assemblies may be contemplated, such as a gimble, rotational mechanism, etc.

The motors 106 may be controlled to provide ultrasonic pulses to the selected well 130 via the transducer 112. An impedance matching circuit 140 may be included to maximize power to the transducer 112. The MPS 100 may also include an amplifier 142, connected to a generator 144, coupled to a high voltage monitor 148. In this example experiment or electronic driving setup, the transducers 112 are driven with sinusoidal pulses amplified by an RF amplifier 142. The impedance matching circuit 140 is used to match the amplifier output to the transducer 112. One to fifty cycle pulses in the 0.5 to 10 MHz range are delivered to the transducer 112 at pulse repetition frequencies of 0.5-5000 Hz. Transducers 112 are driven with peak-to-peak voltages between 0-2 kV, resulting in peak powers approaching 10 kW.

FIG. 6 illustrates a cross-sectional view of a well 130 of the plate 120 of the MPS 100 of FIG. 1. The transducer 112 is arranged below the well 130. The well 130 may be defined within the walls created by the supports 132.

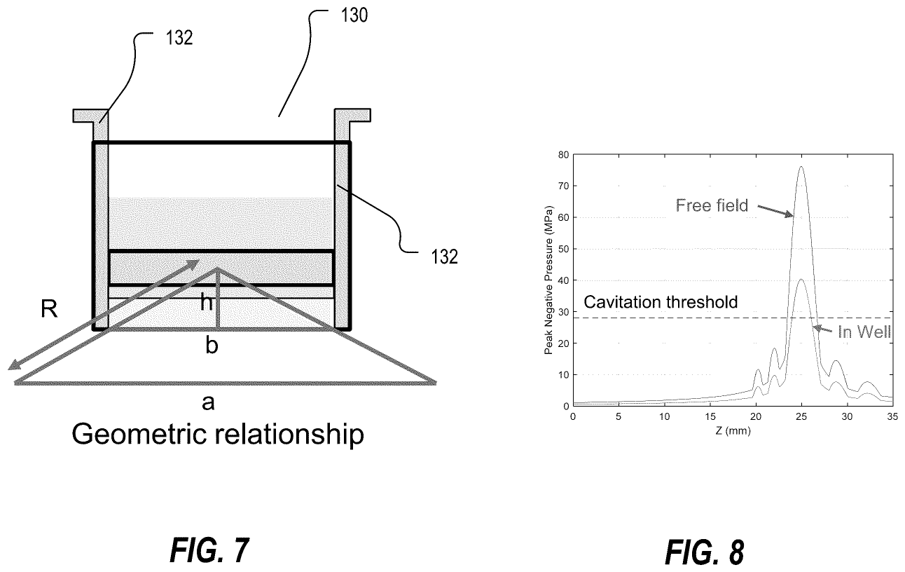

FIG. 7 illustrates a cross-sectional view of the well 130 of the plate 120, similar to FIG. 5, but without the transducer 112. FIG. 6 shows the geometric relationship between the transducer and the well. The aperture (a) and focal distance (R) of the FUS transducer can be designed to focus sound to a prescribed depth (h) into a well 130 given a known well width (b) using a simple geometric relationship. This relationship can be used to optimize the transducer geometry to achieve the requirements for a given MPS layout. The geometric relationship may be defined as:

$$\frac{\sqrt{{( b / 2 )}^{2} + {h}^{2}}}{R} = \frac{b}{a} \qquad Equation 1$$

Alternatively, the transducer matching can be adjusted to correct for any focal shifts. The transducer 112 is comprised of piezoelectric, epoxy and housing materials. The system may be calibrated to account for focal shifts due to propagation through the MPS materials. That is, if there are deviations in focal position after passing through the plate materials below a well (i.e., differences from what is shown in FIG. 7), compensation through calibrations or tuning of the matching media within the reservoir or late materials may be made.

The FUS transducer 112 is arranged below the plate 120 to ensure a high efficiency coupling path between the bottom of the plate 120 and the tissue culture within the plate 120. This allows ultrasound transmission from the transducer 112, through the plate 120 bottom into the tissue culture. The transducer 112 may translate freely in x, y, and z to any well within the plate 120 to facilitate multiple injury experiments on the same platform for increased sample sizes or assessment of different injury conditions. That is, the transducer 112 may perform experiments on some or all of the wells 130.

The FUS waveform emitted from the transducer 112 is coupled to the plate 120 using the medium within the reservoir 122 with material properties that facilitates efficient ultrasound transmission into the tissue layers of the plate 120. As explained above, the coupling medium may be fluid, solid or gel.

The FUS transducer 112 is driven by a high amplitude sinusoidal voltage pulse delivered across the piezoelectric element. Impedance matching circuitry is used to match the amplifier output to the transducer 112. As explained above, 1 to 50 cycle pulses in the 0.5 to 10 MHz range are delivered to the transducer 112 at pulse repetition frequencies of 0.5-5000 Hz. Transducers 112 are driven with peak-to-peak voltages between 0-2 kV resulting in peak powers approaching in the 0-10 kW range. An attenuating circuit can be tapped into the drive line to measure plate reflection and cavitation emission signals received by the transducer 112.

The MPS system 100 is a self-contained, sterile environment enabling continued culture of multicell type neural or other tissue cultures. This allows the assessment of both chronic and acute injury response under physiological conditions. It also facilitates the assessment of the physiological response of repeated injury. Additionally, the system 100 allows simple methods to assess the state of the tissue cultures including but not limited to optical measurements and collection secreted factors associated with injury from the media.

In addition to the above, other applications may be appreciated with the MPS 100. For example, blast model improvements, including refinement of biological platform—the biology will be developed to incorporate multi-cell cultures to better model the physiology of the brain—refinement of blast model hardware—automation for targeting specific biological structures within the platform, improvements to injury confirmation methods; developments for other blast injury models (lung, abdominal organs)—the blast model may be incorporated into other MPS models including but not limited to lung and abdominal organs. Other applications may also include lower pressure FUS applications—low pressure pulses can be used to generate other biological effects including thermal and mechanical effects (hyperthermia, neural modulation); in vitro setup for studying focused ultrasound (FUS) for drug delivery—The platform can be developed for developing low TRL ultrasound facilitated drug delivery methods; in vitro blood brain barrier (BBB) opening test environment—FUS can be used to reversibly open the BBB in humans for drug delivery and disease screening; prophylactics for brain injury—FUS cavitation can be used to develop prophylactics for brain injury in a more general sense (outside TBI); in vitro immuno-mechanical ablation mechanism discovery/development—mechanical ablation has been shown to enhance the immunogenicity in cancer microenvironments in-vivo; material etching for vascularizing tissue cultures: cavitation can be used to bore channels within a tissue scaffold.

Simulations of the FUS transducer 112 integrated with the in-vitro tissue platform show the capacity to focus ultrasound energy into the tissue layers within the wells of the platform. The transducers 112 are designed to achieve sufficient pressure overhead to overcome the pressure lost through the optical and membrane layers of the plate 120 (˜55%) and surpass the cavitation threshold. The size of the focal zone dictates the size of the cavitation zone and can be modulated by increasing the rarefactional pressure amplitude lowering the frequency or blurring the focused sound beam.

FIG. 8 illustrates an example chart for the simulated focal pressure showing distance (mm) vs. pressure (Mpa). The cavitation threshold is a certain acoustic pressure necessary to create bubbles. As illustrated, the extent to which the transducer can generate pressures in excess of the cavitation threshold may vary between the free field and that of the samples within the well 130.

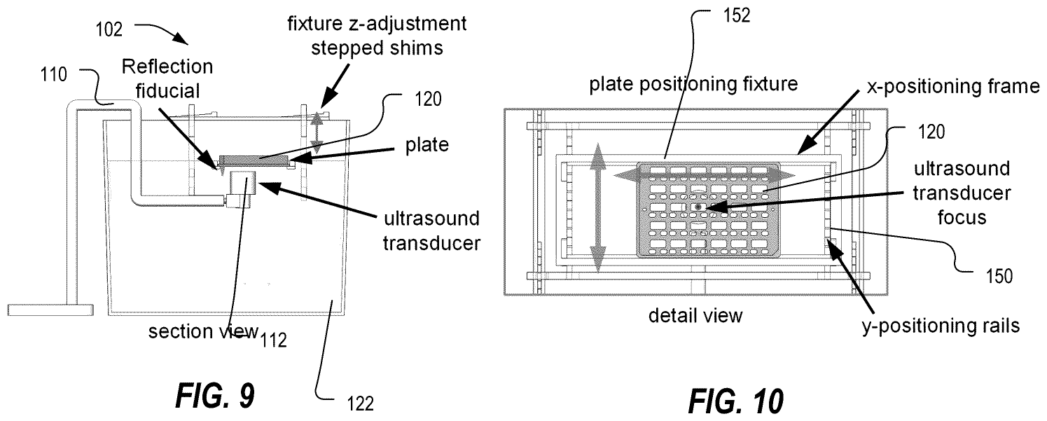

FIG. 9 illustrates a cross-sectional view of a portion of the system 100, including the transducer 112 and plate 120 within the medium 122. FIG. 10 illustrates a top view of the arrangement of FIG. 6 including the MPS 100.

In one example, as described above, the transducer 110 arm may be moved relative to the plate 120 to position the transducer 112 below the respective well 132. In the example shown in FIGS. 9 and 10, the plate 120 may be moved relative to the transducer 112. This may include a frame assembly include a first pair of rails 150 on opposites sides of the plate 120 and a second pair of rails 152 perpendicular to the first pair of rails, the plate 120 configured to move within the medium laterally and axially along each of the rails. Movement along these rails may be manual or motorized. In another example, fine positioning of the transducer within a single well may be done by moving the transducer relative to the plate while coarser well-to-well with respect to the transducer movement may be performed with the plate rail system.

The transducer 112 can be mechanically positioned at different locations within the tissue culture plate 120. Targeting of specific points within a well 130 may be achieved by registering the location of the transducer 112 to the plate 120 using the plate directly or a platform 134 with defined fiducials. One method to achieve this registration is by maximizing the reflected ultrasound signal off the tip of a raised fiducial or minimizing the reflected ultrasound signal within a hole fiducial fixed in a geometrically defined position relative to the plate 120, as explained above. The relationship between the fiducials 136 or 138 and plate 120 can then be used to place the transducer 112 at specific points on the plate 120. Fixturing can be used precisely move the transducer 112 or plate 120 in XYZ to facilitate generating targeted lesions (FIG. 3).

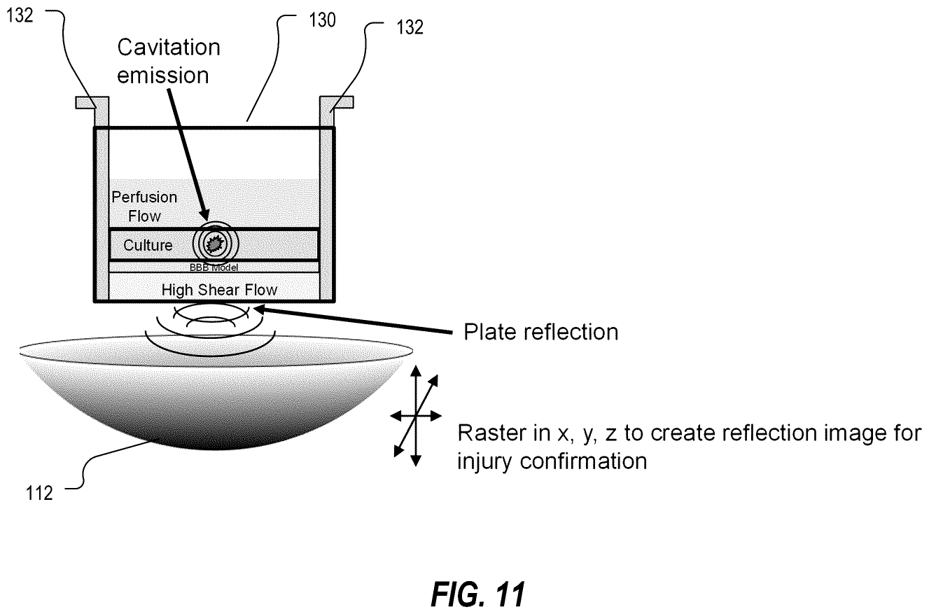

FIG. 11 illustrates a cross-sectional view of the well 130 of the plate 120 of the MPS 100 of FIG. 1, similar to FIG. 6, and includes the plate reflection and cavitation emission. The same FUS transducer 112 used to generate focal cavitation may be used as a ‘passive’ receive transducer to receive reflections from the bottom of the plate 120 or targeting platform 134, as well as the cavitation emission (shock wave) emitted from the cavitation event. The relative timing (Δt) of both signals and sound speed (C) can be used to calculate the depth (d) of the cavitation event within the well. After injury, the same transducer 112 or a higher frequency transducer can be raster scanned in x, y, z to create a reflection image mapping out the injury zone.

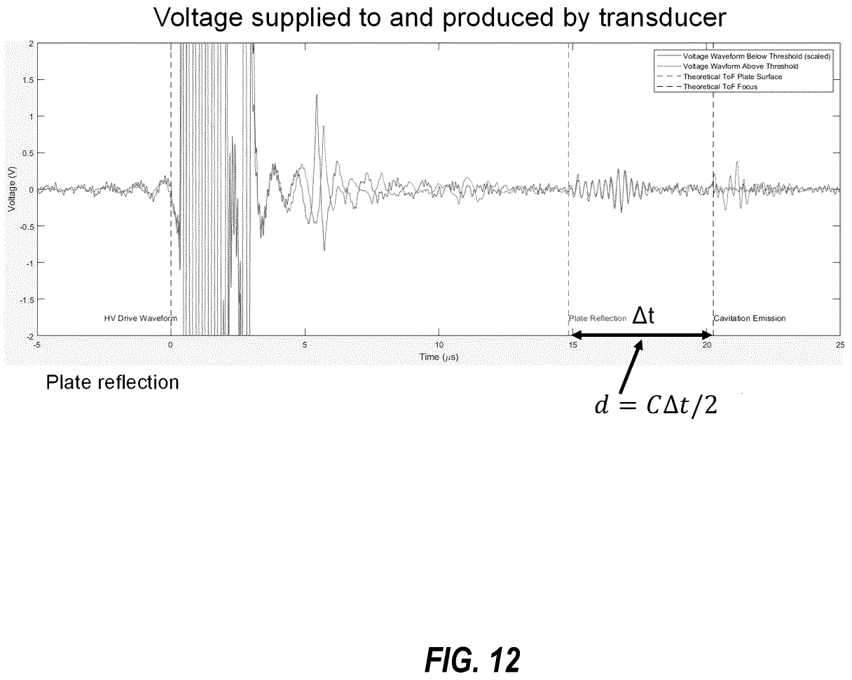

FIG. 12 illustrates an example voltage production of the transducer 112 over time, where,

$$d = C Δ t / 2 \qquad Equation 2$$

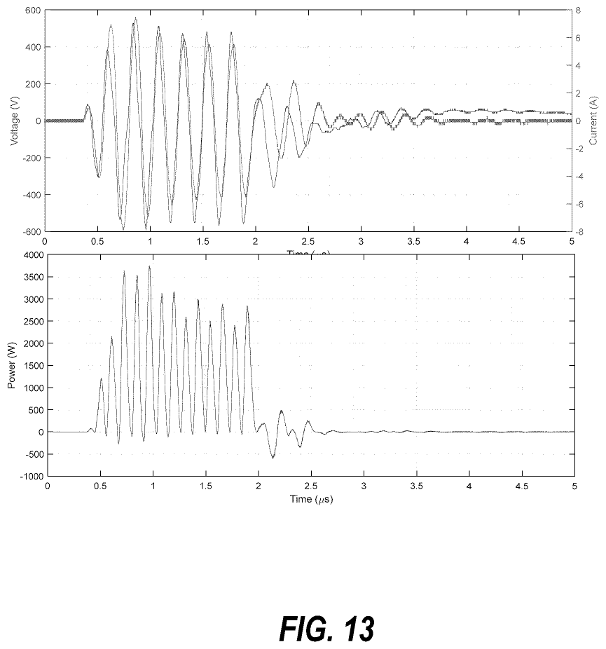

FIG. 13 illustrates an example voltage and power over time of the set up of FIG. 1.

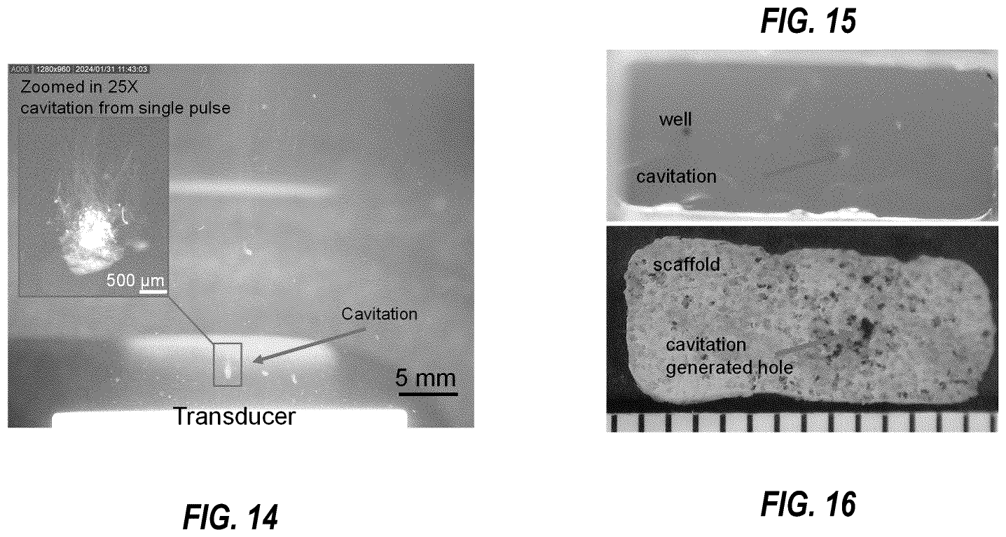

FIG. 14 illustrates a cross-section view of a transducer 112 under excitation, having cavitation. Upon transducer excitation, targeted cavitation zone of approximately 0.5×0.5×1 mm is formed at the focal zone.

FIG. 15 illustrates a view of the transducer 112 being aligned and integrated with the in-vitro plate 120 to generate cavitations within a single well 130 of the plate 120.

FIG. 16 illustrates a view of cavitation generated hole, where, with a sufficient number of pulses, the cavitation zone can fragment a tissue scaffold placed within the well.

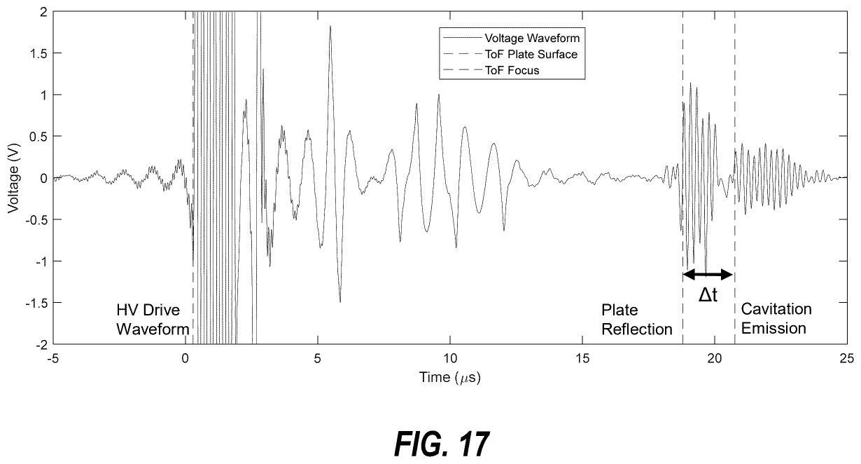

FIG. 17 illustrates an example voltage supplied to the transducer over time of an injury confirmation in situ, where injury confirmation may be confirmed in situ using the signals received by the transducer 112. Injury zones resulting from cavitation were visualized in agarose gel (2%) samples and compared to depth of cavitation calculations performed using the plate reflection and cavitation emission signals received by the transducer 112 (FIG. 20). The depth of the injury zone (d) within the well observed in the agarose matches that performed by the calculation based on receive signal, where taking Equation 3:

$$d = \frac{1481 \frac{m}{s} \times 1.95 μs}{2} = 1.4 mm \qquad Equation 3$$

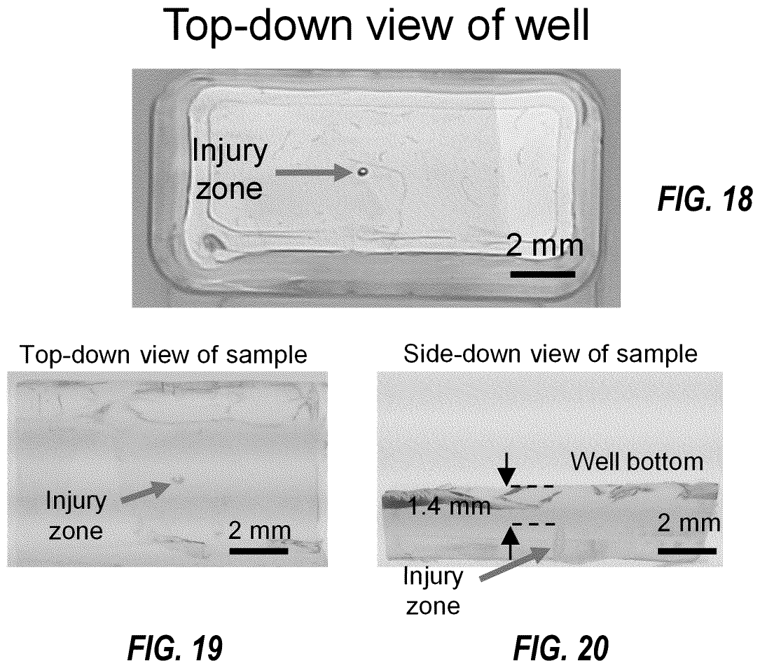

FIG. 18 illustrates a top view of an example well.

FIG. 19 illustrates a top view of a sample.

FIG. 20 illustrates a side view of the sample of FIG. 19. FIGS. 18-20 illustrate the correlation with voltage vs. time signal aligning with FIG. 17 and demonstrate localization feedback. The dT may be used to predict the location of the lesion from a bottom of a specific well where d=C*dt/2 (as shown in FIG. 17).

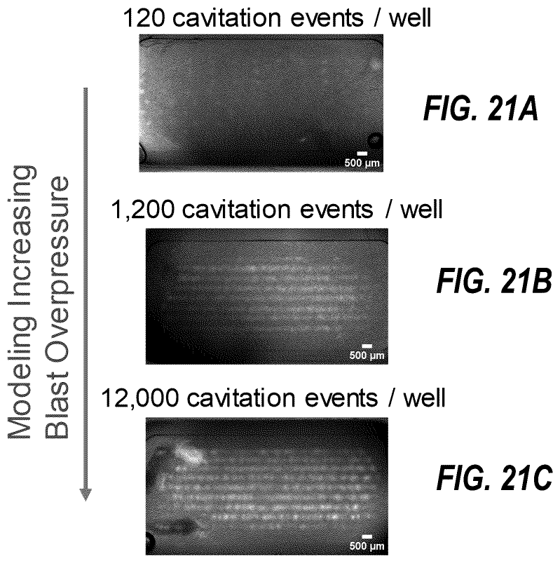

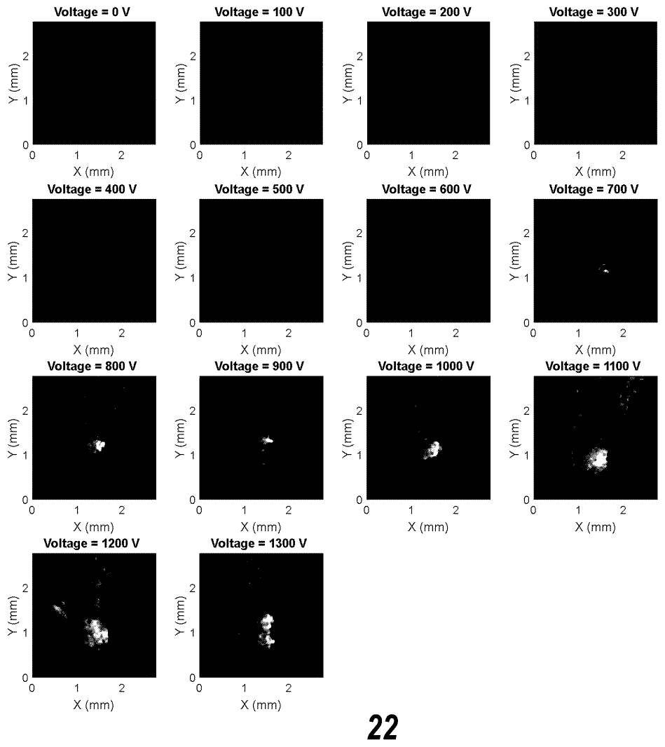

FIGS. 21A-C illustrate example modeling as an increase of overpressure per well 130 where the controller 114 systematically distributes FUS signals across the wells 130. For example, FIG. 21A illustrates a model with 120 cavitation events. FIG. 21B illustrates a model with 1,200 cavitation events. FIG. 21C illustrates a model with 12,000 cavitation events.

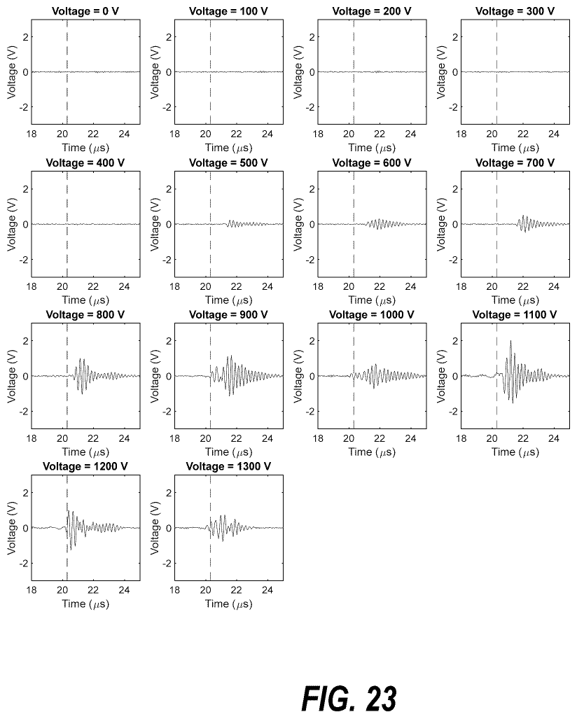

FIG. 22 illustrates a series of focal zone images where the cavitation emissions received by the transducer 112 indicative of the absence or presence of injury. The white masses or blobs are images of cavitation at the focus of the transducer 112. The presence of the white blobs corresponds with the presence of a received signal amplitude in FIG. 23.

FIG. 23 illustrates a series of signals received by the transducer 112 indicative of the absence or presence of injury.

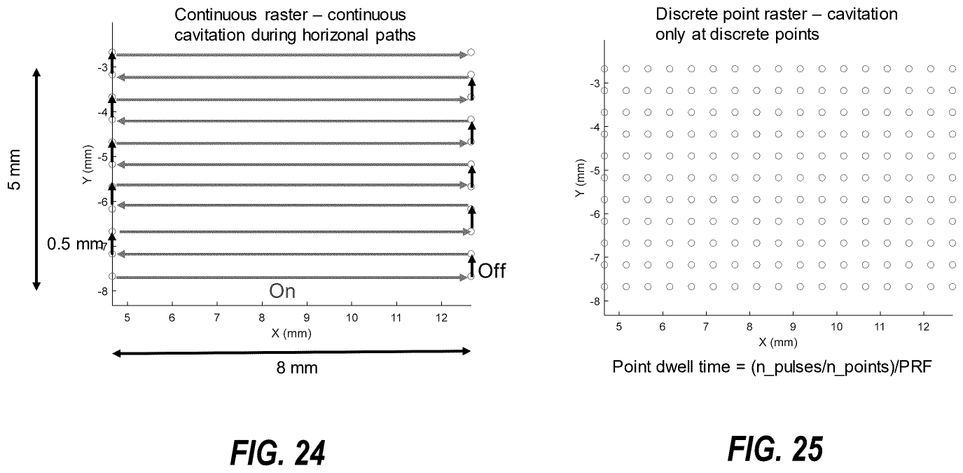

FIG. 24 illustrates an example configuration to deliver pulses of cavitation uniformly across area of tissue using continuous raster. In use, the transducer 112 moves in a raster scanning pattern, line by line, while continuously emitting and receiving signals. In the example shown, the cavitation is applied over horizontal paths where the velocity=total length/n pulses.

FIG. 25 illustrates an example configuration to delivery pulses of cavitation uniformly across area of tissue using discrete points on a grid as opposed to continuous raster scanning. In this example, the point dwell time=n pulses/n points.

FIG. 26 illustrates a cross-sectional view of an example transducer 112. The transducer 112 may include flat piezos with a focusing matching layer. In one example, the transducer 112 may include a curved PZT4, having high strength epoxy and a matching layer made from PerFORM (Somos®).

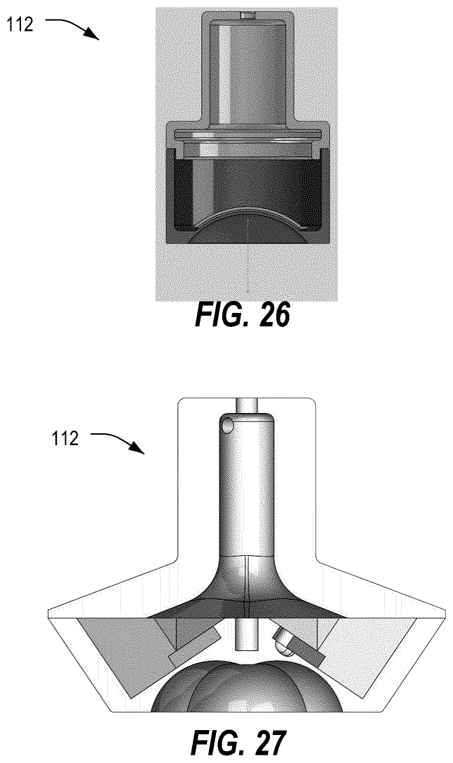

FIG. 27 illustrates a cross-sectional view of another example transducer 112 including a flat APC60, high strength epoxy such as Loctite® E-120 HP, and marine epoxy. The example transducers 112 may provide better energy transfer. Table 1 below illustrates an example chart of the Piezo examples.

TABLE 1

| | | Matching | |
|---|---|---|---|
| | Impedance | Impedance | Shape |
| Piezo | (MRayl) | (MRayl) | Options |
| PZT4 | 30-40 | 7-8 | Flat, curved |
| APC60 | 12-18 | 4-5 | Flat only |

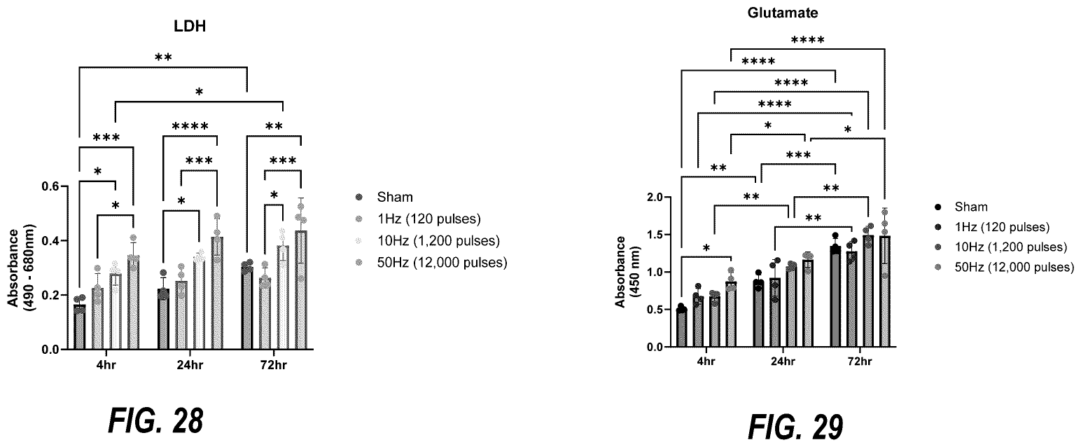

FIGS. 28-37 illustrate results from the experiment set up of FIG. 1.

FIG. 28 illustrates an example chart for a LDH assay blast injury showing absorbance over time.

FIG. 29 illustrates an example chart for Glutamate showing absorbance over time.

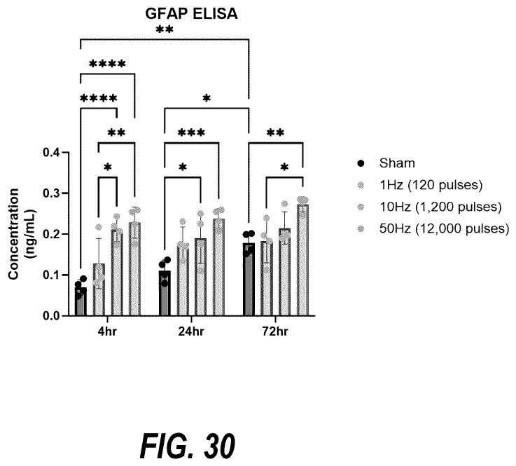

FIG. 30 illustrates an example chart for GFAP showing concentration over time.

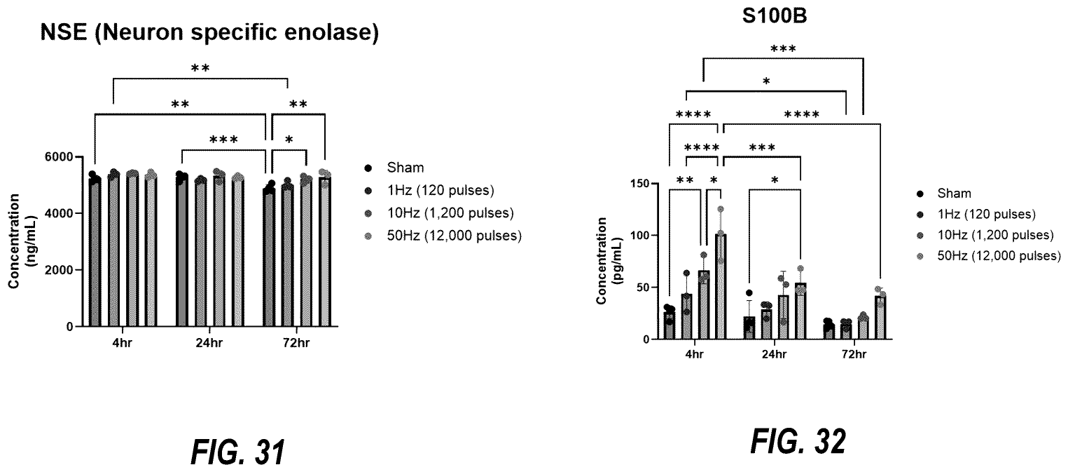

FIG. 31 illustrates an example chart for NSE showing concentration over time.

FIG. 32 illustrates an example chart for S100B showing concentration over time.

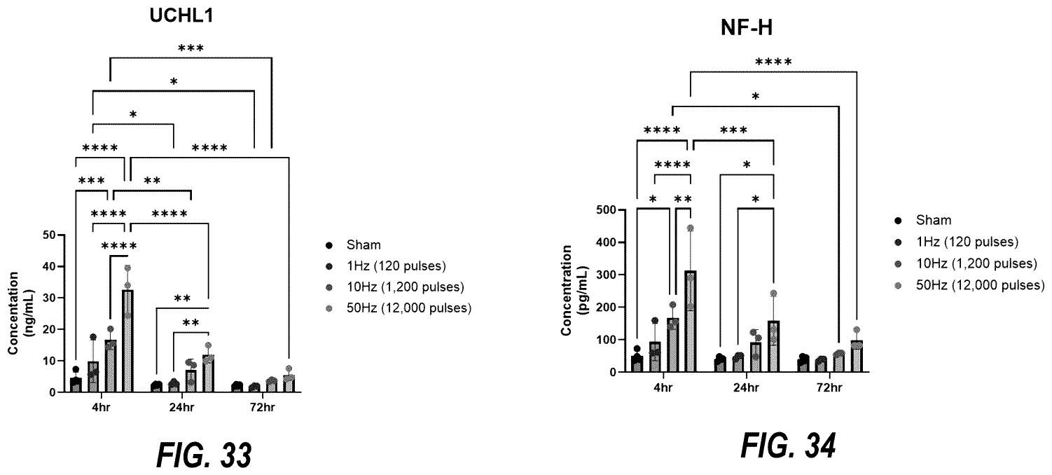

FIG. 33 illustrates an example chart for UCHL showing concentration over time.

FIG. 34 illustrates an example chart for NF-H showing concentration over time.

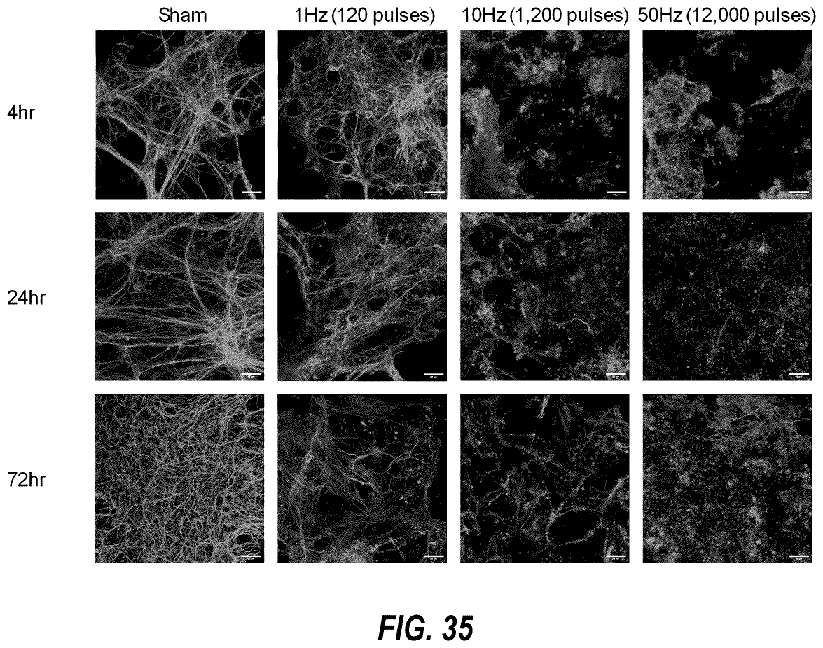

FIG. 35 illustrates example images of neuron structures at various pulse repetition frequencies and doses over time.

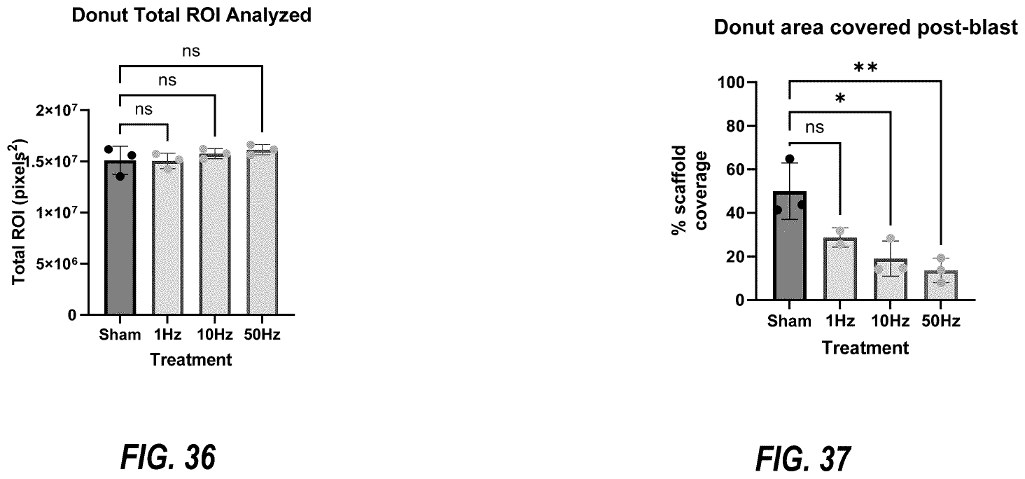

FIG. 36 illustrates an example chart for the total ROI analyzed for neuron networks in scaffolds exposed to blast treatments over frequency.

FIG. 37 illustrates an example chart for the neuron area covered over pulse repetition frequency and dose. This example chart illustrates the effects shown in FIG. 36.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A system to simulate injury in tissue culture, comprising:
a plate configured to:
contain tissue culture and defining a plurality of wells;
a transducer configured to:
couple with the plate to create a coupling path between the plate and the tissue culture within the plate to generate cavitations within the tissue culture;
a medium coupling the transducer to the plate; and
an electronic driver configured to emit a waveform to drive the transducer and allow assessment of the cavitations.

2. The system of claim 1, wherein the wells are arranged in rows and columns defined by supports therebetween.

3. The system of claim 2, wherein a hole is defined at the intersection of the supports to provide a reflective indication of the location of an adjacent well.

4. The system of claim 1, further comprising a metal panel arranged on a first side of the plate between the plate and the transducer, the panel defining a group of openings, each group of openings configured to align with a respective well of the plate.

5. The system of claim 1, wherein the plate is maintained and movable along a rail system, such that the plate is configured to move within the medium laterally and axially with the rail system.

6. The system of claim 5, wherein the movement of the plate is configured to arranged a selected one of the plurality of wells over the transducer to generate cavitations at the selected well.

7. The system of claim 1, wherein the transducer is arranged on a transducer arm, the transducer arm configured to arrange the transducer under a selected one of the plurality of wells to generate cavitations at the selected well.

8. The system of claim 1, wherein the transducer generates cavitations via a waveform and is configured to receive voltage signals indicative of the cavitations in the selected well.

9. The system of claim 1, wherein the transducer is included as part of a stage system having a multi-axis stage control assembly, an arm extending from the stage assembly, and the transducer being arranged on the arm.

10. The system of claim 9, wherein the stage system includes at least one motor configured to drive the control assembly to maneuver the transducer arm.

11. The system of claim 10, further comprising an impedance matching circuit configured to match the output of an RF amplifier to the transducer and maximize power to the transducer.

12. The system of claim 11, further comprising an RF generator and high voltage monitor to monitor for the presence of cavitation.

13. The system of claim 12, wherein the monitoring of cavitation includes at least one of lactate dehydrogenase, glutamate, glial fibrillary acidic protein, neuron specific enolase, S100 calcium-binding protein B, ubiquitin carboxyl-terminal hydrolase L1, neurofilament heavy chain, immunofluorescence imaging or western blot assays of intracellular proteins such as PIEZO1.

14. The system of claim 1, wherein the medium is a liquid.

15. The system of claim 1, wherein the transducer is configured to deliver cavitation uniformly across a well via continuous rastering or discrete point rastering.

16. A system to simulate injury in tissue culture, comprising:
a plate configured to contain tissue culture and defining a plurality of wells;
a transducer arranged with the plate creating a coupling path between the plate and the tissue culture within the plate;
a medium coupling the transducer to the plate; and
a driving system configured to emit a waveform to drive the transducer and allow assessment of injury to the tissue culture.

17. The system of claim 16, further comprising a controller configured to receive cavitation measurements indicated via voltage signals from the transducer.

18. The system of claim 17, wherein the controller provides instructions for the transducer to be arranged below a selected one of the plurality of wells to generate a cavitation at the selected well.

19. The system of claim 17, wherein the controller is configured to determine the position of transducer relative to the well based at least in part on reflective data from the plate.
